(19) United States
(12) Patent Application Publication
(10) Pub. No.: US 2010/0010545 A1
(43) Pub. Date: Jan. 14, 2010

(54) Device having universal coupling linkage for stabilizing vertebrae

(76) Inventors: Gi-Hoon Park, Gimhae-Si (KR); Soo-Jung Moon, Gimhae-Si (KR)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 12/232,952

(22) Filed: Sep. 26, 2008

(30) Foreign Application Priority Data
Jul. 9, 2008 (KR) 10-2008-0066512

(51) Int. Cl. A61B 17/70

(52) U.S. Cl. 606/278; 606/250

(57) ABSTRACT

A device having a universal coupling linkage for stabilizing vertebrae is developed that is comprising: a first link integrally forming a hook-shaped end to engage the connecting rod, a first flat top surface with first thread hole, a sink-down middle portion, an uprising wall in a right angle, an inner flat top surface adjacent to inner end with a central thread hole, a mounting cavity formed at the uprising wall and a circumference edge seat; a second link integrally forming a second hook-shaped end to engage the connecting rod, a second flat top surface adjacent to the outer end with a second thread hole, a sliding rod at opposite end; a swivel forming a plurality of slits for squeezing, a through-hole for inserting the sliding rod to adjust a clearance between the first and second connecting rods; a plurality of plug bolts and thread holes for depressing the connecting rods and the swivel.

### BACKGROUND OF THE INVENTION

1. Field of the invention

The present invention relates to a vertebral stabilizer, and more particularly the device having a set of universal coupling linkage for stabilizing the vertebrae, which can swivel in all directions and adjust the length by extending or retrieving with respect to a swivel joint, which is located in the center of the linkage. Even if a fracture of the vertebrae is the worst situation, e.g. when the vertebrae are severely broken out, the present stabilizer is effectively coupled and supported the worst damaged vertebrae by the swivel operation.

2. Description of the Prior Art

As generally known in the art, degenerative vertebral diseases, including disk degenerative diseases (DDD), spinal stenosis and spondylolisthesis, are subjected to conservative treatment first, and if no improvement occurs, surgical treatment follows. The surgical treatment consists of decompression, fusion (ALIF, PLIF, or posteriolateral fusion), and procedures for stabilizing vertebrae. In some cases, the surgical procedure consists of decompression only, or both decompression and fusion. However, surgery in most cases ends with stabilization. Conventional spinal stabilization uses pedicle screws, connecting rods, and a connecting rod coupling device. Specifically, the universal coupling linkage couples and supports two connecting rods, which are attached by pedicle screws in a line on the left and right sides, to the spinal column. This device serves to reinforce the strength and stability of the entire assembly.

However, it is very difficult to use such a universal coupling linkage during surgery because in most surgical situations, the two connecting rods are not aligned with each other. This requires a universal coupling linkage incorporating a self-adjustment function to deal with misalignment between the connecting rods. However, such a universal coupling linkage incorporating a self-adjustment function includes a number of components that must be assembled and adjusted during surgery, which lengthens the surgery time and increases the difficulty.

In an attempt to solve aforementioned problems, a universal coupling linkage is capable of accommodating the misalignment of connecting rods.

FIG. 1 is a perspective view of conventional coupling device. The conventional coupling device includes a first hook coupling member 11 having a first hook 11A for engaging one of the connecting rods, a second hook coupling member 12 having a second hook 12A for engaging the other connecting rod, a cross-rod 13, and locking members 14 for coupling the first and second coupling members 11 and 12 to the left- and right-side cross-rods 13, respectively.

Specifically, the locking members 14 couple the coupling members 11 and 12 while being able to roll and slide with regard to the cross-rods 13. The conventional construction has to ensure that the connecting rods are aligned each other. Somehow, the coupling members 11 and 12 can be coupled to the cross-rod 13 with difficulty by rolling or sliding with regard to the cross-rod 13.

However, the conventional coupling device has the following problem. The locking members 14 are continuously loaded a predetermined level of force in order to fasten the coupling members 11 and 12 to the cross-rod 13. If the fastening force is excessive, it is troublesome to slightly roll or slide the coupling members 11 and 12 during the surgery. On the contrast, if the fastening force is too weak, it causes the connecting rods are loosened to couple and be insufficiently supported.

In addition, if the fracture of the vertebra is severe, the conventional coupling device has big troublesome to fix the vertebrae, especially in the middle point of the fracture. In other words, the conventional coupling device is not applicable to the worst fracture of the vertebrae.

### SUMMARY OF THE INVENTION

Accordingly, the universal coupling linkage of the present invention has developed to solve the aforementioned problems that are occurring in the conventional device. The present invention adopts a swivel, a first link and second link components and a plurality of locking members for stabilizing vertebrae, which has a simple construction for swiveling and adjusting the clearance between the connecting rods.

In accordance with an aspect of the present invention, the assembled first and second links is engaged between a pair of connecting rods for stabilizing the vertebrae. The device is comprised of that: a first link component (110) is integrally forming a first hook-shaped end (111) for engaging a first connecting rod (R), a first outer flat top surface to have a first thread hole (112), a sink down middle portion, an uprising wall portion (113W) in a right angle, and an inner flat top surface to have a central thread hole (114), wherein the uprising wall portion (113W) has formed a mounting cavity (113) with an inlet (113) and an outlet, and a hemispherical circumference edge seat (113A) formed at the outlet.

A second link component (120) is integrally forming a second hook-shaped end (121) for engaging a second connecting rod (R), a second outer flat top surface to have a second thread hole (122), and a sliding rod (123) at inner end.

A swivel (130) is forming a plurality of slits (132) being extended from outlet of the insertion hole (131) for expanding or contracting when it is depressed, a through-hole (131) for inserting and passing through the sliding rod (123) of the second link (120) to adjust a clearance between the first and second connecting rods (R).

A plurality of plug bolts (141, 142, 143) is inserted to the first, central and second plug bolt holes (112, 114, 122), respectively, the plug bolts inwardly rotated for depressing the first, second connecting rods (R) and the swivel (130), so that the rotation of the plug bolt will be compressed to lock the swivel (130) and the sliding rod (123).

The inner flat top surface of the inner end (110B) is protruded toward to the second component (120) for acting as a stopper to prevent excessive tilt of the sliding rod (123).

The plurality of plug bolts (141, 142, 143) and the plug bolt holes (112, 122, 114) has formed a male thread (141A, 142A, 143A) and female thread (112A, 122A, 114A), respectively to be threaded inward or outward for acting as a locking member.

### BRIEF DESCRIPTION OF THE DRAWINGS

FIG. 1 is a perspective view of a conventional coupling rod for supporting the connecting rods.

FIG. 2 is a perspective view of a set of the universal coupling linkage for stabilizing the vertebrae according to the present invention.

FIG. 3 is an exploded perspective view illustrating each component of the universal coupling linkage for stabilizing the vertebrae according to the present invention.

FIG. 4 is a cross-sectional view showing each component of the universal coupling linkage for stabilizing the vertebrae according to the present invention.

FIGS. 5 to 7 are cross-sectional views showing the operation of the universal coupling linkage for stabilizing the vertebrae according to the present invention.

FIG. 5 shows a locking of the universal coupling linkage for stabilizing the vertebrae according to the present invention.

FIG. 6 shows a releasing of the universal coupling linkage for stabilizing the vertebrae according to the present invention.

FIG. 7 shows a swiveling of the universal coupling linkage for stabilizing the vertebrae according to the present invention.

FIG. 8 is an enlarged cross-sectional view showing a swivel of the universal coupling linkage for stabilizing the vertebrae according to the present invention.

FIG. 9 shows an application of the universal coupling linkage for stabilizing the vertebrae according to the present invention.

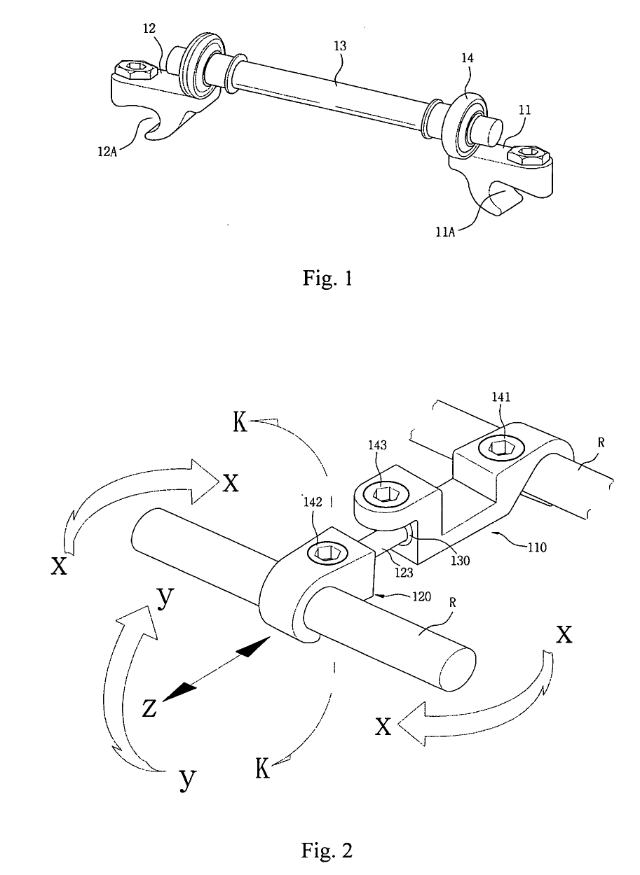

### DESCRIPTION OF THE PREFERRED EMBODIMENTS

Hereinafter, an embodiment of the universal coupling linkage of the present invention will be described in detail with reference to the accompanying drawings. The same or similar components are used the same reference numerals to designate in the following description and drawings, so it is avoided the duplicated or repeated description for the same or similar components.

FIG. 2 shows a set of the universal coupling linkage for stabilizing the vertebrae. As shown in FIG. 2, the universal coupling linkage of the present invention is comprised of a first link component 110 having a first hook for engaging a connecting rod R positioned on a right-side, and a second link component 120 having a second hook for engaging the other connecting rod R positioned on the opposite side. The first and second components 110 and 120 are linked to each other through a spherical swivel 130, so that the linkage can move or swivel any direction. Especially, the second link component 120 has integrally formed a sliding rod 123 extended to the opposite end to slide along with the axial direction (Z direction). The swivel joint make the linkage to rotate in K direction, tilt or swivel in any directions (in X and Y directions) with regard to the swivel joint.

Such a movement of the swiveling, extending or retrieving of the present invention ensures the stabilizing system properly connected for stabilizing the vertebrae, even though the connecting rods R are not aligned each other. Even if the vertebrae are severely fractured or broken out, the swiveling operation of the universal coupling linkage will guarantee to easily and properly connect between the connecting rods R.

The unique configuration of the present invention, i.e.; the universal coupling linkage adopts the swivel 130 for connecting between the first and second link components 110 and 120, which is capable to swivel and adjust the length by extending, retrieving. Such an operation guarantees the proper connection between two connecting rods R, even if the fracture of the bones is severe situation.

The respective component of the universal coupling linkage for vertebral stabilization according to the present invention will now be described in detail.

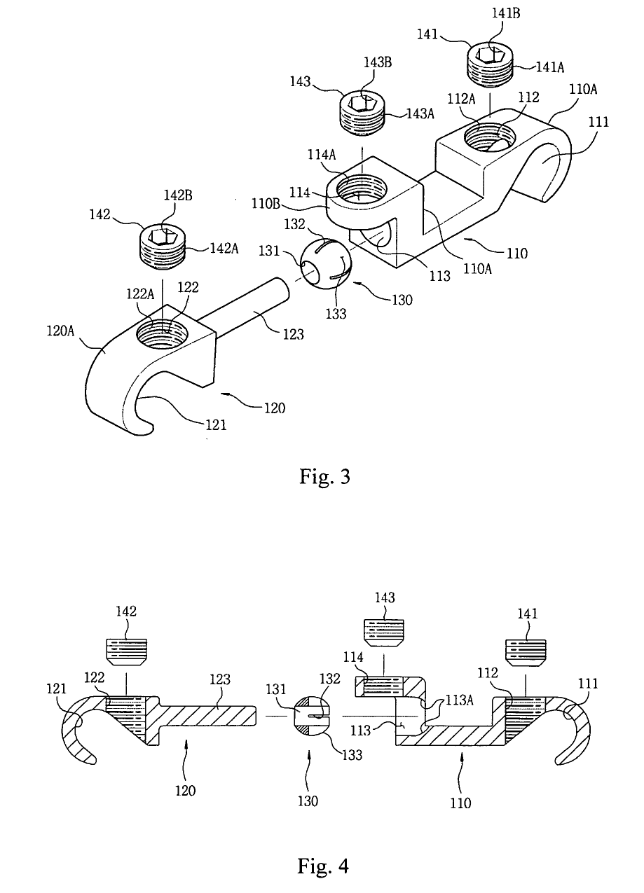

FIG. 3 is an exploded perspective view showing the construction of the universal coupling linkage for vertebral stabilization according to the present invention, and FIGS. 4 to 8 are cross-sectional views showing the operation and function of the universal coupling linkage for vertebral stabilization.

As shown, the inventive device is comprised of a first link component 110, a second link component 120, a spherical-shaped swivel 130 with an inserting hole 131, a pair of plug bolts 141, 142, and a central plug bolt 143. The first link component 110 has a first hook-shaped end 111 integrally formed on the outer end 110A to engage with a connecting rod R. The first link component 110 has a flat top on the outer end 110A, adjacent to the first hook-shaped end 111 to provide a drilled thread hole 112 forming a female thread at the inner wall thereof. Then, the locking plug bolt 141 is inserted to the drilled thread hole 112 for fastening the connecting rod R, which is engaged with the first hook-shaped end 111. The plug bolt 141 and the thread hole 112 have the same pitch of threads 141A and 112A for locking the connecting rod (R).

The inner end of the first link component 110 has formed a raising wall portion 113W and a flat top surface 114 in a right angle direction. The upraising wall 113W has formed a mounting cavity 113 for extending out the sliding rod 123. The mounting cavity 113 has formed a hemispherical circumference edge seat 113A at the outlet thereof, so that the diameter of the circumferential edge seat 113A is gradually decreases from the inner diameter of the mounting cavity 113. Therefore, the spherical swivel 130 will be maintained inside of the mounting cavity 113. The inner end 110B of the first link component 110, which is located opposite side of the outer end 110A, has an upraised wall 113W and a flat top surface 114 being bent in a right angle with respect to the outer end 110A. The upraised wall 113W has formed a mounting cavity 113 for mounting the swivel 130. The flat top surface 114 of the inner end 110B is protruded toward to the second component 120 for acting as a stopper to prevent the excessive tilt of the sliding rod 123. The flat top surface 114 of the inner end 110B has also formed a central thread hole 114, of which the center line is intersected in perpendicular to that of the mounting cavity 113. A central thread hole 114 has a female thread 114A for threading the central plug bolt 141 to be rotated inward or outward as acting the locking member. The central plug bolt 143 and the central thread hole 114 have formed the same pitch of male and female threads 143A and 114A, respectively. The central plug bolt 143 has a hexagonal recess 143B for a hexagonal wrench same as the first and second plug bolts 141 and 142 having the hexagonal recesses 141B and 142B, respectively.

According to the aforementioned construction, the inward rotation of the central plug bolt 143 in the central thread hole 114 will exert the frictional forces on the swivel 130, which is mounted in the mounting cavity 113, against the circumferential edge seat 113A. On contrast, the outward rotation of the central plug bolt 143 releases the depressing force of swivel 130 and the circumferential edge seat 113A.

The second link component 120 has also formed a hook-shaped end 121 on the outer end 120A to engage with a second connecting rod R. The second link component 120 has also formed a second thread hole 122 on the flat top surface adjacent to the outer end for inserting a second plug bolt 142. The second thread hole 122 of the second link component 120 has formed a female thread at the inner wall. The second plug bolt 142 has formed a male thread 142A for mating with the female thread 122A of the second thread hole 122. Then, the second plug blot 142 is inserted into the second thread hole 122 for fastening the left connecting rod R, which is engaged with the hook-shaped end 121.

Further, the second link component 120 has integrally formed a sliding rod 123, which is extended to the opposite direction from the second outer end 120A. Then, the sliding rod 123 is inserted through the mounting cavity 113 of the first link component 110. Particularly, the sliding rod 123 is installed to be extended through the mounting cavity 113 of the first component 110, while it is inserted into the insertion hole 131 of the swivel 130. According to the present configuration, the sliding bar 123 is installed through the mounting cavity 113 to coincide with the first link component 110. Therefore, the universal coupling linkage of the present invention can slide to adjust the length by extending or retrieving.

The swivel 130 is adopted to join the first and second link components 110 and 120 under the free loading condition while the sliding rod is freely sliding inward or outward. The swivel 130 has an insertion hole 131 for inserting through the sliding bar 123 of the second component 120. The swivel 130 has a plurality of slits 132 extended from the outlet of the insertion hole 131, so that the insertion hole 131 can be contracted when it is depressed. The overall shape of the swivel 130 forms a sphere, so that it can smoothly swivel while the linkage is installed on the connecting rods. Then, the sliding rod is inserted into the insertion hole 131 via the mounting cavity 113 of the first component 110. However, it is also possible to grind the contacting part of the swivel 130 to mount on the circumferential edge seat 113A of the first component 110. Once the swivel 130 is firmly depressed, the first and second link components 110 and 120 are locked to each other.

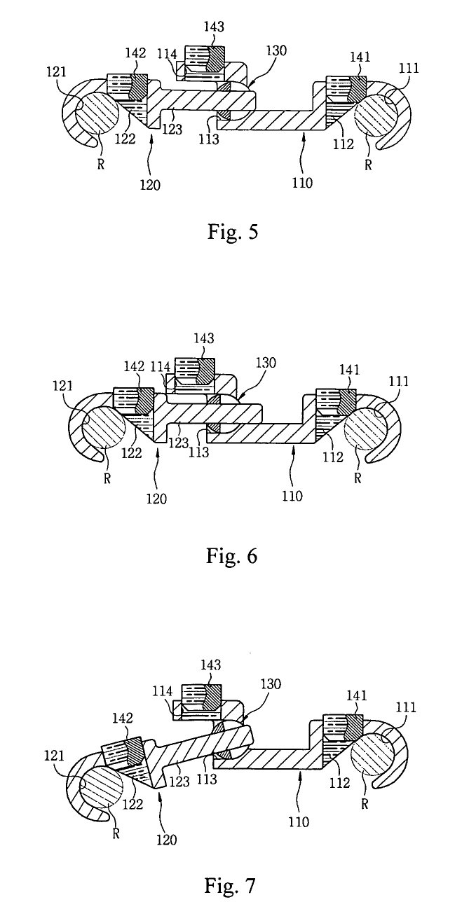

Such a configuration, the swivel 130 is mounted inside the mounting cavity 113 of the first link component 110 while the sliding rod 123 of the second link component 120 is passing through the insertion hole 131 of the swivel 130. If the swivel 130 is not depressed by the central plug bolt 143, the swivel 130 allows the sliding rod 123, which is passed through the insertion hole 131, freely sliding inward or outward. As a result, the universal coupling linkage device of the present invention can adjust the clearance between the connecting rods by extending or retrieving the sliding rod 123. The linkage is also possible to be bent and rotated about the swivel 130. As seen in FIG. 6, the sliding rod 123 has pushed inward from the situation shown in FIG. 5, and the overall length has shortened. The swivel 130 can also freely rotate (or swivel) inside the mounting cavity 113 as long as it is not depressed by the central plug bolt 143. As a result, the first and second link components 110 and 120 can be freely bent with regard to the swivel 130. As seen in FIG. 7, the universal coupling linkage allows such a bending operation from the situation as seen in FIG. 5.

Once the swivel 130 is depressed by the central plug bolt 143 to exert the force against the circumferential edge seat 113A, which is formed on the circumferential edge of the mounting cavity 113, the swivel 130 is constrained so that the swivel will be locked inside the mounting cavity 113. In addition, the insertion hole 131 of the swivel 130 and the sliding rod 123, which has extended through the insertion hole 131, are depressed and constrained by the central plug bolt 143. As a result, the first and second link components 110, 120 are locked not to allow any movements (i.e., swiveling sliding forward, retrieving, or tilting) with respect to the swivel 130. In other words, the universal coupling linkage of the present invention is prevented from any operational movement when it is locked.

The universal coupling linkage according to the present invention has employed a simply configuration of a swivel joint to couple the first and second link components 110 and 120 for moving all directions, such as an extending, retrieving, swiveling or bending suitable to fit the vertebral surgery. As a result, the universal coupling linkage is easily engaged between the two connecting rods R in any situation.

Hereinafter, the operation and function of the universal coupling linkage for fixing the vertebrae according to the present invention will be described in detail with reference to the accompanying drawings.

FIG. 4 is the disassembly of the universal coupling linkage showing the first and second link components 110, 120 and the swivel 130. The sliding rod 123 of the second link component 120 is inserted through the insertion hole 131 of swivel 130. With the inserted condition, the swivel 130 is placed into the mounting cavity 113 of the first component 110.

The central plug bolt 143 is positioned to the central thread hole 114, and fastened by rotating inward direction until a bottom of the central plug bolt 143 is slightly stick-out in front inlet of the mounting cavity 113. Therefore, the swivel 130 is prevented to fall off from the mounting cavity 113. However, the central plug bolt 143 is not depressed to lock the swivel 130 at this moment.

The first and second plug bolts 141 and 143 are positioned in the first and second plug bolt holes 112, 122, respectively. The first and second plug bolts 141, 143 are inserted into the first and second plug bolt holes 112, 122 for ready to be fastened, when the first and second hook-shaped end 111, 121 are engaged to the connecting rods R without troublesome.

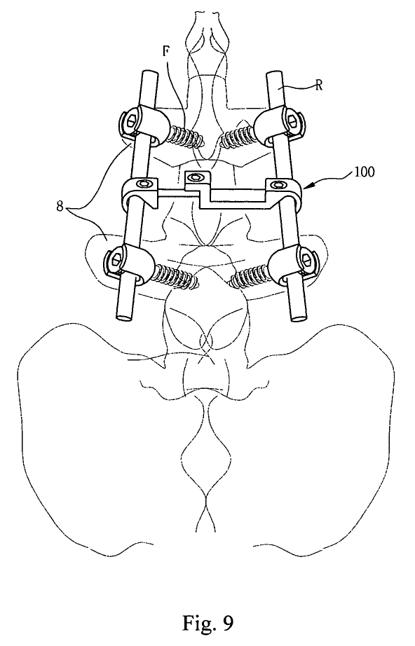

Then, the assembly of the first and second link components 110, 120, which is joined via the swivel 130 as seen in FIG. 5, is engaged to the left and right connecting rods R, which are installed on the left and right of the spinal column. As shown in FIG. 9, it is assumed that a number of pedicle screws F have already been installed on the vertebrae 8 of the left and right spinal column. Then, the connecting rods R, which are installed to connect the pedicle screws F on the left and right respectively, have arranged to align along with the longitudinal direction of the spinal column.

The first hook-shaped end 111 located at the first outer end 110A of first component 110 engages to the left connecting rod R, and the second hook-shaped end 121 located at the outer end 120A of second component 120 engages to the right connecting rod R.

In order to adjust the clearance between the left and right connecting rods R, the second link component 120 is simply pulled or pushed against the first link component 110, i.e. the sliding rod is inwardly extended or retrieved in Z direction as shown in FIG. 2. When the central plug bolt 143 is not depressed, the sliding rod 123 of the second link component 120 is free to slide inward or retrieve through the insertion hole 131 of the swivel 130, so that the length of the universal coupling device can adjust the clearance between the left and right connecting rods R. Referring to FIG. 5 and FIG. 6, the sliding rod 123 has moved from the maximum length to the minimum length.

In addition referring to FIG. 2 and FIG. 7, if the first and second link components 110, 120 is tilted (or rotated in K direction) with respect to the swivel 130, the sliding rod 123, which is inserted through the insertion hole 131 of the swivel 130, is adjusted the length and the angle of tilt at the same time. Thus, the universal coupling device is bending and sliding to accommodate the misalignment between the connecting rods R.

Even if, the fracture of vertebrae is the worst situation, the universal coupling linkage has capability to manage the worst damaged fractures by adjusting the length and the tilting angle of the first and second link components 110, 120 with respect to the swivel 130. Thus, the clearance between the left and right connecting rods R is controlled to avoid the interference of the fractured vertebrae. The swivel 130 is smoothly rotated (swiveled) inside of the mounting cavity 113, so that the universal coupling device can be tilting and sliding to avoid the interference with the fractured vertebrae. FIG. 7 shows a bending (tilting) operation of the universal coupling linkage device.

The first and second plug bolts 141, 142 are rotated inward direction for exerting a frictional force on the connecting rods R against the threads of the first and second plug bolt holes 111, 121 to constrain the connecting rods R.

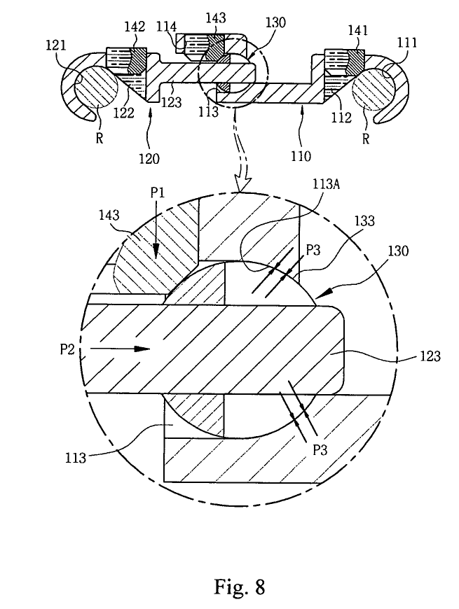

After the connecting rods R are constrained in the manner of described above, the central plug bolt 143 is finally fastened by rotating inward to the central thread hole 114. Then, the front tip of the central plug bolt 143 is contacted to the spherical surface of the swivel 130 and exerted a frictional force P1 to the direction as shown in FIG. 8. The frictional force P1 applied to the swivel 130 is converted to an axial trust force P2 acting in the axial direction of the sliding rod 123. As a resultant of the interacting forces between the front tip of the central plug bolt 143 and the spherical surface of the swivel 130, the swivel 130 is pushed toward the circumferential edge seat 113A in the mounting cavity 113. The front end 133 of the swivel 130 is tightly pushed to exert the trusting force against the circumferential edge seat 113A of the first link component 110, which will be constrained and immobilized the swivel 130. As a result, the first and second link components 110, 120 are locked, so that the linkage is no longer movable with respect to the swivel 130. The swivel 130 is compressed due to the reaction force P3 acting between the front end 133 of the swivel 130 and the circumferential edge seat 113A. Therefore, the sliding rod 123 of the second link component 120, which is extended by passing through the insertion hole 131 of the swivel 130, is locked. So, the swivel 130 can not rotate in the mounting cavity 113. At the same time, the sliding rod 123 can not move inward or outward through the insertion hole 131 of the swivel 130.

As a result, the left and right connecting rods R are firmly coupled and supported by the universal coupling linkage according to the present invention, as shown in FIG. 9.

The universal coupling linkage for stabilizing the vertebrae according to the present invention has an advantage of swivel operation to be properly positioned, even though the fractured vertebrae is severe, the device can be easily adjusted to support two connecting rods without troublesome.

The universal coupling linkage can be easily swiveled, extended and retrieved for engaging the left and right connecting rods R by adjusting the angle and clearance, regardless the misalignment.

The swivel has formed a plurality of slits from outlet of the insertion hole to the near center, which is capable to constrain or release in the radial direction, so that the sliding bar can be easily locked and released to slide through the insertion hole of the swivel.

The inner end of the first component is protruded to act as a stopper for limiting the tilt angle of the sliding bar.

Although an embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A device having a universal coupling linkage for stabilizing vertebrae, the device comprising:
a first link component (110) integrally forming a first hook-shaped end (111) for engaging a first connecting rod (R), a first flat top surface adjacent to an outer end (110A) to have a first thread hole (112), a sink-down middle portion, an uprising wall portion in a right angle, and an inner flat top surface adjacent to an inner end (110B) to have a central thread hole (114), wherein said uprising wall portion has formed a mounting cavity (113) with an inlet (113) and an outlet thereof, and a hemispherical circumference edge seat (113A) formed at the outlet,
a second link component (120) integrally forming a second hook-shaped end (121) for engaging a second connecting rod (R), a second flat top surface adjacent to the outer end (120A) to have a second thread hole (122), and a sliding rod (123) at opposite end,
a swivel (130) forming a plurality of slits (132) for squeezing or expanding when it is depressed, a through-hole (131) for inserting through the sliding rod (123) of the second link (120) to adjust a clearance between the first and second connecting rods (R), and
a plurality of plug bolts (141, 142, 143) for inserting to thread holes (112, 114, 122), respectively, said plug bolts inwardly rotated for depressing the connecting rods (R) and the swivel (130), so that the rotation of the plug bolt will be compressed to lock the swivel (130) and the sliding rod (123).

2. The device for stabilizing vertebrae as claimed in claim 1, wherein said inner flat top surface adjacent to the inner end (110B) is protruded toward to the second link component (120) for acting as a stopper to prevent excessive tilt of the sliding rod (123).

3. The device for stabilizing vertebrae as claimed in claim 1, wherein said plurality of plug bolts and the thread holes are first, central, and second plug bolts (141, 142, 143) and first, central, and second thread holes (112, 114, 122), which are forming same pitches of male threads (141A, 143A, 142A) and female threads (112A, 114A, 122A), respectively.

4. The device for stabilizing vertebrae as claimed in claim 3, wherein said central plug bolt (143) is inserted into the central thread hole (114) for fastening to exert a frictional force on a spherical surface of the swivel (130) against the circumferential edge seat (113A).

5. The device for stabilizing vertebrae as claimed in claim 1, wherein said plurality of slits (132) of the swivel (130) is formed from an outlet of the through-hole (131) to two-third diameter of the swivel (130).
